(19) United States
(12) Patent Application Publication
(10) Pub. No.: US 2012/0220799 A1
(43) Pub. Date: Aug. 30, 2012

(54) NOVEL PROCESS

(75) Inventors: Abhay Gaitonde, Panvel (IN); Chitra Vaidya, Panvel (IN); P. Khairnar, Panvel (IN)

(73) Assignee: Generics [UK] Limited, Hertfordshire (GB)

(21) Appl. No.: 13/468,620

(22) Filed: May 10, 2012

Related U.S. Application Data
(62) Division of application No. 12/373,396, filed on Apr. 15, 2009, now abandoned.

(30) Foreign Application Priority Data
Jul. 12, 2006 (IN) 1107/MUM/2006

(51) Int. Cl. C07C 227/34; C07C 229/08; C07C 227/12

(52) U.S. Cl. 562/401; 562/553

(57) ABSTRACT

The present invention relates to a novel process for the preparation of γ-amino acids, such as (±)-3-(aminomethyl)-5-methyl-hexanoic acid 1, which is a key intermediate in the preparation of the potent anticonvulsant pregabalin, (S)-(+)-3-(aminomethyl)-5-methyl-hexanoic acid 2.

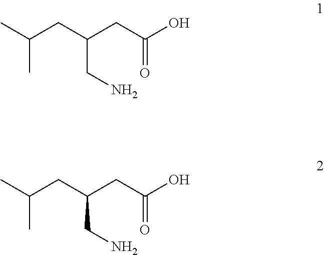

### FIELD OF THE INVENTION

The present invention relates to a novel process for the preparation of γ-amino acids, such as (±)-3-(aminomethyl)-5-methyl-hexanoic acid 1, which is a key intermediate in the preparation of the potent anticonvulsant pregabalin, (S)-(+)-3-(aminomethyl)-5-methyl-hexanoic acid 2.

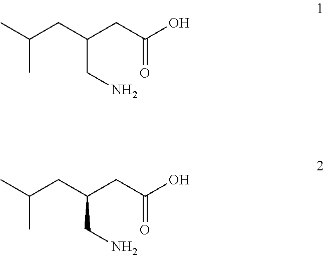

### BACKGROUND OF THE INVENTION

(±)-3-(aminomethyl)-5-methyl-hexanoic acid, or (±) β-isobutyl-γ-amino-butyric acid, or (±) isobutyl-GABA, hereafter called racemic pregabalin 1, was first reported in Synthesis, 1989, 953. The synthetic process reported involved the addition of nitromethane to an ethyl 2-alkenoate and the nitro ester thus formed was reduced using palladium on carbon. Subsequent hydrolysis using hydrochloric acid afforded racemic pregabalin as the hydrochloride salt. The free base of racemic pregabalin 1 was then prepared by ion exchange chromatography.

An alternative process reported in U.S. Pat. No. 5,637,767 describes the condensation of isovaleraldehyde with diethyl malonate. The 2-carboxy-2-alkenoic acid thus formed was reacted with a cyanide source, specifically potassium cyanide. The cyano diester product was decarboxylated by heating with sodium chloride in DMSO and water, and hydrolyzed using KOH to give the potassium salt of a cyano acid. This was hydrogenated in situ using sponge nickel and neutralized with acetic acid to give racemic pregabalin 1.

A further process for preparing racemic pregabalin hydrochloride has been reported in US patent application 20050043565. This process involved a Wittig-Horner reaction between isovaleraldehyde and triethyl phosphonoacetate to give the ethyl 2-alkenoate. Addition of nitromethane using TBAF, followed by hydrogenation using Raney nickel afforded the lactam, which was hydrolyzed using HCl to form the hydrochloride salt of the amino acid.

The present inventors investigated preparing racemic pregabalin 1 by the most convenient and shortest route, which also avoids using hazardous and environmentally unsuitable reagents. The process reported in U.S. Pat. No. 5,637,767 uses highly toxic KCN, which should be avoided. Also, the use of sponge nickel could be potentially hazardous. The route reported in US 20050043565 gives the hydrochloride salt instead of the free base. It is well known that there are practical difficulties in the isolation of amino acids from aqueous media, due to the formation of zwitterionic species. The formation of the HCl salt of racemic pregabalin 1 necessitates an aqueous work-up, which leads to poor yields and lengthy work-up procedures.

Definitions

For the purposes of the present invention, an “alkyl” group is defined as a monovalent saturated hydrocarbon, which may be straight-chained or branched, or be or include cyclic groups. An alkyl group may optionally be substituted, and may optionally include one or more heteroatoms N, O or S in its carbon skeleton. Preferably an alkyl group is straight-chained or branched. Preferably an alkyl group is not substituted. Preferably an alkyl group does not include any heteroatoms in its carbon skeleton. Examples of alkyl groups are methyl, ethyl, n-propyl, i-propyl, n-butyl, i-butyl, t-butyl, n-pentyl, cyclopentyl, cyclohexyl and cycloheptyl groups. Preferably an alkyl group is a C₁₋₁₂ alkyl group, preferably a C₁₋₆ alkyl group. Preferably a cyclic alkyl group is a C₃₋₁₂ cyclic alkyl group, preferably a C₅₋₇ cyclic alkyl group.

An “alkenyl” group is defined as a monovalent hydrocarbon, which comprises at least one carbon-carbon double bond, which may be straight-chained or branched, or be or include cyclic groups. An alkenyl group may optionally be substituted, and may optionally include one or more heteroatoms N, O or S in its carbon skeleton. Preferably an alkenyl group is straight-chained or branched. Preferably an alkenyl group is not substituted. Preferably an alkenyl group does not include any heteroatoms in its carbon skeleton. Examples of alkenyl groups are vinyl, allyl, but-1-enyl, but-2-enyl, cyclohexenyl and cycloheptenyl groups. Preferably an alkenyl group is a C₂₋₁₂ alkenyl group, preferably a C₂₋₆ alkenyl group. Preferably a cyclic alkenyl group is a C₃₋₁₂ cyclic alkenyl group, preferably a C₅₋₇ cyclic alkenyl group.

An “alkynyl” group is defined as a monovalent hydrocarbon, which comprises at least one carbon-carbon triple bond, which may be straight-chained or branched, or be or include cyclic groups. An alkynyl group may optionally be substituted, and may optionally include one or more heteroatoms N, O or S in its carbon skeleton. Preferably an alkynyl group is straight-chained or branched. Preferably an alkynyl group is not substituted. Preferably an alkynyl group does not include any heteroatoms in its carbon skeleton. Examples of alkynyl groups are ethynyl, propargyl, but-1-ynyl and but-2-ynyl groups. Preferably an alkynyl group is a C₂₋₁₂ alkynyl group, preferably a C₂₋₆ alkynyl group.

An “aryl” group is defined as a monovalent aromatic hydrocarbon. An aryl group may optionally be substituted, and may optionally include one or more heteroatoms N, O or S in its carbon skeleton. Preferably an aryl group is not substituted. Preferably an aryl group does not include any heteroatoms in its carbon skeleton. Examples of aryl groups are phenyl, naphthyl, anthracenyl and phenanthrenyl groups. Preferably an aryl group is a C₄₋₁₄ aryl group, preferably a C₆₋₁₀ aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

An optionally substituted alkyl, alkenyl, alkynyl, aryl, arylalkyl, arylalkenyl, arylalkynyl, alkylaryl, alkenylaryl or alkynylaryl group may be substituted with one or more halo, alkylhalo, hydroxy, thio, nitro, amino, alkyl, alkoxy or carboxy group. Any optional substituent may be protected. Suitable protecting groups for protecting optional substituents are known in the art, for example from “Protective Groups in Organic Synthesis” by T. W. Greene and P. G. M. Wuts (Wiley-Interscience, 3^{rd} edition, 1999).

An “alkoxy” group is defined as a —O-alkyl group.

A “halo” group is a fluoro, chloro, bromo or iodo group.

An “alkylhalo” group is an alkyl group substituted with one or more halo group.

A “hydroxy” group is a —OH group. A “thio” group is a —SH group. A “nitro” group is a —NO₂ group. An “amino” group is a —NH₂ group. A “carboxy” group is a —CO₂H group.

The γ-amino acids of the present invention have at least one chiral centre and therefore exist in at least two stereoisomeric forms. For the purposes of the present invention, a γ-amino acid is “racemic” if it comprises the two stereoisomers in a ratio of from 60:40 to 40:60, preferably in a ratio of about 50:50. A γ-amino acid is “enantiomerically enriched”, if it comprises 70% or more of only one stereoisomer, preferably 80% or more, preferably 90% or more. A γ-amino acid is “enantiomerically pure”, if comprises 95% or more of only one stereoisomer, preferably 98% or more, preferably 99% or more, preferably 99.5% or more, preferably 99.9% or more

For the purposes of the present invention, a γ-amino acid is “substantially free” of lactam impurity, if it comprises less than 3% lactam impurity, preferably less than 2%, preferably less than 1%, preferably less than 0.5%, preferably less than 0.1%.

### SUMMARY OF THE INVENTION

A first aspect of the present invention provides a process of preparing a γ-amino acid 11, comprising the step of deprotecting the ester and reducing the nitro functionality of a γ-nitro ester 16 in one step to afford the γ-amino acid 11:

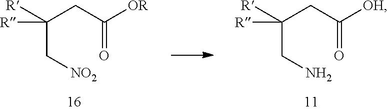

wherein R is any group that can be removed under the same reducing conditions that can convert a nitro group to an amino group, and wherein R′ and R″ are independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, or both R′ and R″ together with the carbon atom to which they are attached from a cyclic alkyl or cyclic alkenyl group, each of which may optionally be substituted, and each of which may optionally include one or more heteroatoms N, O or S in its carbon skeleton. Preferably the γ-amino acid 11 is racemic.

Aliphatic nitro groups like those in γ-nitro ester 16 can be reduced to amine groups by many reducing agents including catalytic hydrogenation (using hydrogen gas and a catalyst such as Pt, Pt/C, PtO₂, Pd, Pd/C, Rh, Ru, Ni or Raney Ni); Zn, Sn or Fe and an acid; AlH₃—AlCl₃; hydrazine and a catalyst; [Fe₃(CO)₁₂]-methanol; TiCl₃; hot liquid paraffin; formic acid or ammonium formate and a catalyst such as Pd/C; LiAlH₄; and sulfides such as NaHS, (NH₄)₂S or polysulfides.

Likewise, esters like those in γ-nitro ester 16 can be deprotected or hydrolysed to give the free carboxylic acids under a number of conditions. Preferred esters, such as benzyl, carbobenzoxy (Cbz), trityl (triphenylmethyl), benzyloxymethyl, phenacyl, diphenylmethyl and 4-picolyl esters, can be deprotected by catalytic hydrogenolysis (using hydrogen gas and a catalyst such as Pt, Pt/C, PtO₂, Pd, Pd/C, Rh, Ru, Ni or Raney Ni). Many of these preferred esters can also be deprotected under acidic conditions (using, for example, CH₃CO₂H, CF₃CO₂H, HCO₂H, HCl, HBr, HF, CH₃SO₃H and/or CF₃SO₃H); under basic conditions (using, for example, NaOH, KOH, Ba(OH)₂, K₂CO₃ or Na₂S); by catalytic transfer hydrogenolysis (using a hydrogen donor such as cyclohexene, 1,4-cyclohexadiene, formic acid, ammonium formate or cis-decalin and a catalyst such as Pd/C or Pd); by electrolytic reduction; by irradiation; using a Lewis acid (such as AlCl₃, BF₃, BF₃-Et₂O, BBr₃ or Me₂BBr); or using sodium in liquid ammonia. Benzyl esters can also be deprotected using aqueous CuSO₄ followed by EDTA; NaHTe in DMF; or Raney Ni and Et₃N. Carbobenzoxy esters can also be deprotected using Me₃SiI; or LiAlH₄ or NaBH₄ and Me₃SiCl. Trityl esters can also be deprotected using MeOH or H₂O and dioxane. Phenacyl esters can also be deprotected using Zn and an acid such as AcOH; PhSNa in DMF; or PhSeH in DMF.

Thus, preferably, R is a benzyl, carbobenzoxy (Cbz), trityl, benzyloxymethyl, phenacyl, diphenylmethyl or 4-picolyl group, each of which may optionally be substituted. If substituted, R may be substituted with one or more nitro, halo, alkyl or alkoxy groups.

Preferably, R is a benzyl, substituted benzyl, carbobenzoxy (Cbz), substituted carbobenzoxy (Cbz) or trityl group. Preferably, R is a benzyl group; the benzyl group may be substituted with one or more nitro, halo or alkyl groups, in one or more ortho, meta or para positions. Preferred substituted benzyl groups are p-nitrobenzyl, o-nitrobenzyl, p-methoxybenzyl, p-bromobenzyl, 2,4,6-trimethylbenzyl and 2,4-dimethoxybenzyl.

Preferably, R′ and R″ are independently hydrogen or an alkyl group, or both R′ and R″ together with the carbon atom to which they are attached from a cyclic alkyl group. Preferably, R′ and R″ are independently hydrogen or a C₁₋₆ alkyl group, or both R′ and R″ together with the carbon atom to which they are attached from a C₅₋₇ cyclic alkyl group. In one preferred embodiment, one of R′ and R″ is hydrogen and the other is i-butyl. In another preferred embodiment, both R′ and R″ together with the carbon atom to which they are attached from a cyclohexyl group.

Preferably, the deprotection of the ester and the reduction of the nitro functionality are carried out using hydrogen gas in the presence of a catalyst, preferably Pd/C, Pt/C or PtO₂, preferably Pd/C. Other methods known to the person skilled in the art involving known reagents, catalysts and solvents can be used to perform this one step deprotection and reduction, for example, hydrogenolysis with other catalysts such as Raney nickel or the use or ammonium formate with a catalyst such as Pd/C.

Preferably, the γ-amino acid 11 is obtained in a yield of 60% or more, preferably 65% or more, preferably 70% or more. Preferably, the γ-amino acid 11 is obtained substantially free of lactam impurity.

Preferably, the γ-nitro ester 16 is obtained by reacting an unsaturated ester 15 with nitromethane:

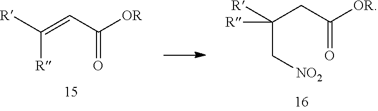

Preferably, the unsaturated ester 15 is converted into the γ-nitro ester 16 by reaction with nitromethane in the presence of a base. The base can be an organic base such as a trialkyl amine or an inorganic base such as a carbonate, a hydroxide or a hydrogen carbonate. A particularly preferred base is DBU.

Preferably, the γ-nitro ester 16 is obtained in a yield of 50% or more, preferably 55% or more, preferably 60% or more.

Preferably, the unsaturated ester 15 is obtained by reacting an aldehyde or ketone 14 with a phosphonoacetate:

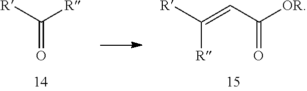

Preferably, aldehyde or ketone 14 is reacted with the phosphonoacetate in the presence of a base. The base can be an organic base such as a trialkyl amine or an inorganic base such as a carbonate, a hydroxide or a hydrogen carbonate. A particularly preferred base is potassium carbonate.

Preferably, the unsaturated ester 15 is obtained in a yield of 70% or more, preferably 80% or more, preferably 90% or more, preferably 95% or more.

Preferably, the phosphonoacetate 9 is prepared in situ from a trialkyl phosphite 8 and an acetic acid ester 3:

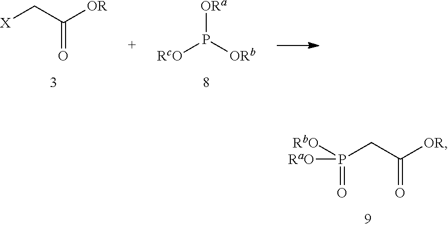

wherein X is a leaving group, and R^{a}, R^{b} and R^{c} are independently alkyl groups.

Preferably, the leaving group X is a halo or sulfonate group. When X is a halo group, it may be a chloro, bromo or iodo group, preferably a bromo group. When X is a sulfonate group, it may be a mesylate, triflate, tosylate or besylate group.

Preferably, the phosphonoacetate 9a is prepared in situ from triethyl phosphite 8a and benzyl bromoacetate 3a:

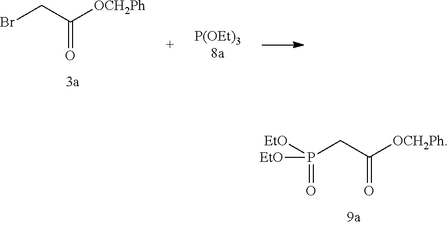

If R′ and R″ are not the same and the γ-amino acid 11 is racemic, then the process of the first aspect of the present invention may further comprise the step of resolving the racemic γ-amino acid 11 to provide an enantiomerically pure or enantiomerically enriched γ-amino acid. The resolution can be done by following well-established and reported routes. For example, U.S. Pat. No. 5,637,767, which is herein incorporated by reference in its entirety, reports the resolution of racemic pregabalin 1 to pregabalin 2 by selective crystallisation with (S)- or (R)-mandelic acid.

Preferably, the unsaturated ester 15, the γ-nitro ester 16, the racemic and the resolved γ-amino acid 11 are obtained on a commercial scale, preferably in batches of 1 kg or more, 10 kg or more, 100 kg or more, 500 kg or more, or 1000 kg or more.

A second aspect of the present invention provides a racemic γ-amino acid, when prepared by a process of the first aspect of the present invention. The second aspect of the present invention also provides an enantiomerically pure or enantiomerically enriched γ-amino acid, when prepared by a process of the first aspect of the present invention.

A third aspect of the present invention provides a racemic γ-amino acid, substantially free of lactam impurity. The third aspect of the present invention also provides an enantiomerically pure or enantiomerically enriched γ-amino acid, substantially free of lactam impurity. By lactam impurity is meant lactam 17 obtained by an intra-molecular condensation reaction:

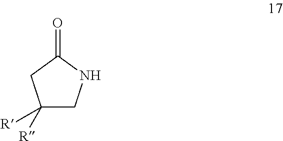

A fourth aspect of the present invention provides a pharmaceutical composition comprising the γ-amino acid of the second or third aspect of the present invention.

A fifth aspect of the present invention provides use of the γ-amino acid of the second or third aspect of the present invention for the manufacture of a medicament for the treatment of epilepsy, pain, neuropathic pain, cerebral ischemia, depression, psychoses or anxiety. The fifth aspect also provides a method of treating or preventing epilepsy, pain, neuropathic pain, cerebral ischemia, depression, psychoses or anxiety, the method comprising administering a therapeutically of prophylactically effective amount of the γ-amino acid of the second or third aspect of the present invention to a patient in need thereof. Preferably the patient is a mammal, preferably a human.

A sixth aspect of the present invention provides a process of preparing racemic pregabalin 1, comprising the step of deprotecting the ester and reducing the nitro functionality of a 3-nitromethyl-5-methyl-hexanoic acid ester 6 in one step to afford racemic pregabalin 1:

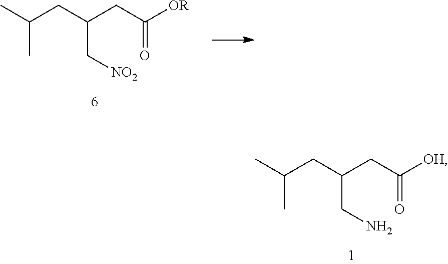

wherein R is any group that can be removed under the same reducing conditions that can convert a nitro group to an amino group.

Aliphatic nitro groups like those in 3-nitromethyl-5-methyl-hexanoic acid ester 6 can be reduced to amine groups by many reducing agents including catalytic hydrogenation (using hydrogen gas and a catalyst such as Pt, Pt/C, PtO₂, Pd, Pd/C, Rh, Ru, Ni or Raney Ni); Zn, Sn or Fe and an acid; AlH₃—AlCl₃; hydrazine and a catalyst; [Fe₃(CO)₁₂]-methanol; TiCl₃; hot liquid paraffin; formic acid or ammonium formate and a catalyst such as Pd/C; LiAlH₄; and sulfides such as NaHS, (NH₄)₂S or polysulfides.

Likewise, esters like those in 3-nitromethyl-5-methyl-hexanoic acid ester 6 can be deprotected or hydrolysed to give the free carboxylic acids under a number of conditions. Preferred esters, such as benzyl, carbobenzoxy (Cbz), trityl (triphenylmethyl), benzyloxymethyl, phenacyl, diphenylmethyl and 4-picolyl esters, can be deprotected by catalytic hydrogenolysis (using hydrogen gas and a catalyst such as Pt, Pt/C, PtO₂, Pd, Pd/C, Rh, Ru, Ni or Raney Ni). Many of these preferred esters can also be deprotected under acidic conditions (using, for example, CH₃CO₂H, CF₃CO₂H, HCO₂H, HCl, HBr, HF, CH₃SO₃H and/or CF₃SO₃H); under basic conditions (using, for example, NaOH, KOH, Ba(OH)₂, K₂CO₃ or Na₂S); by catalytic transfer hydrogenolysis (using a hydrogen donor such as cyclohexene, 1,4-cyclohexadiene, formic acid, ammonium formate or cis-decalin and a catalyst such as Pd/C or Pd); by electrolytic reduction; by irradiation; using a Lewis acid (such as AlCl₃, BF₃, BF₃-Et₂O, BBr₃ or Me₂BBr); or using sodium in liquid ammonia. Benzyl esters can also be deprotected using aqueous CuSO₄ followed by EDTA; NaHTe in DMF; or Raney Ni and Et₃N. Carbobenzoxy esters can also be deprotected using Me₃SiI; or LiAlH₄ or NaBH₄ and Me₃SiCl. Trityl esters can also be deprotected using MeOH or H₂O and dioxane. Phenacyl esters can also be deprotected using Zn and an acid such as AcOH; PhSNa in DMF; or PhSeH in DMF.

Thus, preferably, R is a benzyl, carbobenzoxy (Cbz), trityl, benzyloxymethyl, phenacyl, diphenylmethyl or 4-picolyl group, each of which may optionally be substituted. If substituted, R may be substituted with one or more nitro, halo, alkyl or alkoxy groups.

Preferably, R is a benzyl, substituted benzyl, carbobenzoxy (Cbz), substituted carbobenzoxy (Cbz) or trityl group. Preferably, R is a benzyl group; the benzyl group may be substituted with one or more nitro, halo or alkyl groups, in one or more ortho, meta or para positions. Preferred substituted benzyl groups are p-nitrobenzyl, o-nitrobenzyl, p-methoxybenzyl, p-bromobenzyl, 2,4,6-trimethylbenzyl and 2,4-dimethoxybenzyl.

Preferably, the deprotection of the ester and the reduction of the nitro functionality are carried out using hydrogen gas in the presence of a catalyst, preferably Pd/C, Pt/C or PtO₂, preferably Pd/C. Other methods known to the person skilled in the art involving known reagents, catalysts and solvents can be used to perform this one step deprotection and reduction, for example, hydrogenolysis with other catalysts such as Raney nickel or the use or ammonium formate with a catalyst such as Pd/C.

Preferably, the racemic pregabalin 1 is obtained in a yield of 60% or more, preferably 65% or more, preferably 70% or more. Preferably, the racemic pregabalin 1 is obtained substantially free of lactam impurity.

Preferably, the 3-nitromethyl-5-methyl-hexanoic acid ester 6 is obtained by reacting an ester of 5-methyl-2-hexenoic acid 5 with nitromethane:

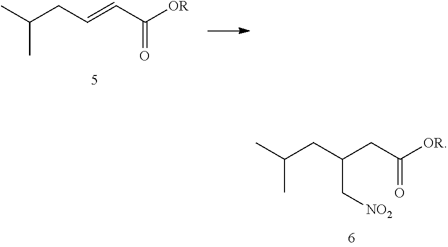

Preferably, the 5-methyl-2-hexenoic acid ester 5 is converted into the 3-nitromethyl-5-methyl-hexanoic acid ester 6 by reaction with nitromethane in the presence of a base. The base can be an organic base such as a trialkyl amine or an inorganic base such as a carbonate, a hydroxide or a hydrogen carbonate. A particularly preferred base is DBU.

Preferably, the 3-nitromethyl-5-methyl-hexanoic acid ester 6 is obtained in a yield of 50% or more, preferably 55% or more, preferably 60% or more.

Preferably, the 5-methyl-2-hexenoic acid ester 5 is obtained by reacting isovaleraldehyde 4 with a phosphonoacetate:

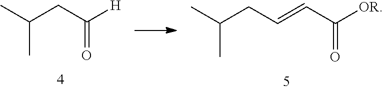

Preferably, isovaleraldehyde 4 is reacted with the phosphonoacetate in the presence of a base. The base can be an organic base such as a trialkyl amine or an inorganic base such as a carbonate, a hydroxide or a hydrogen carbonate. A particularly preferred base is potassium carbonate.

Preferably, the 5-methyl-2-hexenoic acid ester 5 is obtained in a yield of 70% or more, preferably 80% or more, preferably 90% or more, preferably 95% or more.

Preferably, the phosphonoacetate 9 is prepared in situ from a trialkyl phosphite 8 and an acetic acid ester 3:

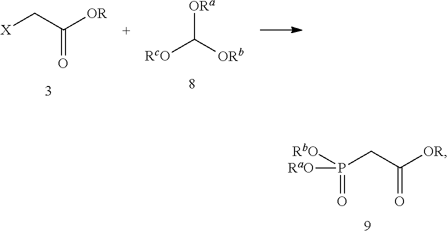

wherein X is a leaving group, and R^{a}, R^{b} and R^{c} are independently alkyl groups.

Preferably, the leaving group X is a halo or sulfonate group. When X is a halo group, it may be a chloro, bromo or iodo group, preferably a bromo group. When X is a sulfonate group, it may be a mesylate, triflate, tosylate or besylate group.

Preferably, the phosphonoacetate 9a is prepared in situ from triethyl phosphite 8a and benzyl bromoacetate 3a:

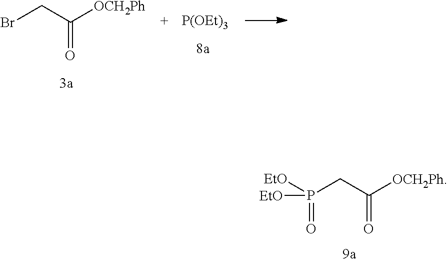

A preferred embodiment of the sixth aspect of the present invention is illustrated in scheme 1.

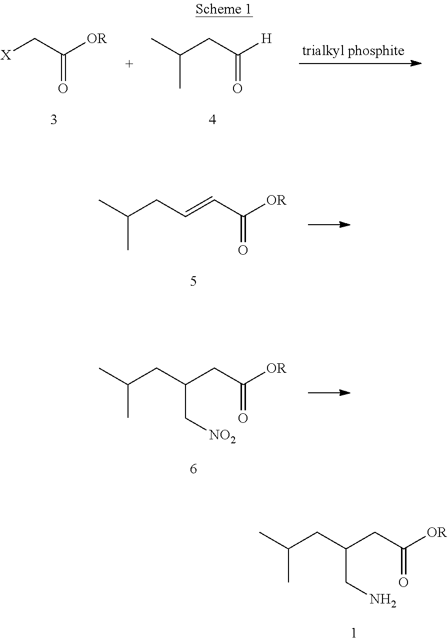

A seventh aspect of the present invention provides racemic pregabalin 1, when prepared by a process of the sixth aspect of the present invention.

An eighth aspect of the present invention provides a process of preparing pregabalin 2, wherein the process comprises the process of preparing racemic pregabalin 1 of the sixth aspect of the present invention. The conversion of racemic pregabalin 1 to pregabalin 2 can be done by following well-established and reported routes of resolution. For example, U.S. Pat. No. 5,637,767, which is herein incorporated by reference in its entirety, reports the resolution of racemic pregabalin 1 to pregabalin 2 by selective crystallisation with (S)- or (R)-mandelic acid.

A ninth aspect of the present invention provides pregabalin 2, when prepared by a process of the eighth aspect of the present invention.

Preferably, the 5-methyl-2-hexenoic acid ester 5, the 3-nitromethyl-5-methyl-hexanoic acid ester 6, the racemic pregabalin 1 and the pregabalin 2 are obtained on a commercial scale, preferably in batches of 1 kg or more, 10 kg or more, 100 kg or more, 500 kg or more, or 1000 kg or more.

A tenth aspect of the present invention provides a pharmaceutical composition comprising pregabalin 2 of the ninth aspect of the present invention.

An eleventh aspect of the present invention provides use of pregabalin 2 of the ninth aspect of the present invention for the manufacture of a medicament for the treatment of epilepsy, pain, neuropathic pain, cerebral ischemia, depression, psychoses or anxiety. The eleventh aspect also provides a method of treating or preventing epilepsy, pain, neuropathic pain, cerebral ischemia, depression, psychoses or anxiety, the method comprising administering a therapeutically of prophylactically effective amount of pregabalin 2 of the ninth aspect of the present invention to a patient in need thereof. Preferably the patient is a mammal, preferably a human.

A twelfth aspect of the present invention provides racemic pregabalin substantially free of lactam impurity.

A thirteenth aspect of the present invention provides pregabalin substantially free of lactam impurity.

A fourteenth aspect of the present invention provides a pharmaceutical composition comprising pregabalin substantially free of lactam impurity.

A fifteenth aspect of the present invention provides use of pregabalin, substantially free of lactam impurity, for the manufacture of a medicament for the treatment of epilepsy, pain, neuropathic pain, cerebral ischemia, depression, psychoses or anxiety. The fifteenth aspect also provides a method of treating or preventing epilepsy, pain, neuropathic pain, cerebral ischemia, depression, psychoses or anxiety, the method comprising administering a therapeutically of prophylactically effective amount of pregabalin, substantially free of lactam impurity, to a patient in need thereof. Preferably the patient is a mammal, preferably a human.

In the context of the twelfth to fifteenth aspects of the present invention, by lactam impurity is meant lactam 7 obtained by an intra-molecular condensation reaction:

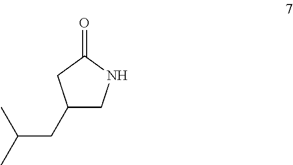

### DETAILED DESCRIPTION OF THE INVENTION

First, the inventors attempted to follow the route as reported in Synthesis, 189, 953. 5-Methyl-2-hexenoic acid ethyl ester was prepared by a Wittig-Horner reaction on isovaleraldehyde according to the procedure reported in US 20050043565. Addition of nitromethane was carried out using DBU as the base. The nitro group was then reduced by bubbling hydrogen gas in the presence of palladium on carbon. The product obtained was the lactam 7, which was hydrolyzed using HCl to give the HCl salt of racemic pregabalin. Ion-exchange chromatography, however, gave the free base 1 contaminated to a large extent by the lactam 7.

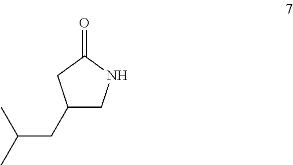

Then, the sequence of the steps was changed to avoid the troublesome formation of the lactam 7. The hydrolysis of the ester was carried out prior to the reduction of the nitro functionality. The ester group was hydrolyzed using lithium hydroxide in THF-water. The nitro acid was successfully hydrogenated to racemic pregabalin 1. No trace of lactam was seen. The yield of isolated amino acid 1 was between 25-30%. The advantage of this route over that reported in Synthesis was that the isolation of the amino acid 1 was by mere crystallization from 2-propanol. No cumbersome ion-exchange chromatography was required. This is very important for the commercial production of this product.

Therefore the present invention relates to a process of preparing a γ-amino acid, comprising the steps of deprotecting or hydrolysing the ester functionality of a γ-nitro ester to afford a γ-nitro acid, followed by reducing the nitro functionality of the γ-nitro acid to afford the γ-amino acid. Preferably the ester hydrolysis is carried out using a base, such as lithium hydroxide. Preferably the nitro functionality is reduced by catalytic hydrogenation using, for example, hydrogen gas and palladium on carbon.

In order to increase the yield of hydrogenation and also reduce the number of steps, the inventors explored the idea of using an alternative group instead of the ethyl group for protection of the carboxylic acid. When a group such as a benzyl or substituted benzyl ester was used, it was found that subsequent hydrogenation deprotected the ester and reduced the nitro group, enabling a one-pot conversion to the amino acid 1.

Also, it was observed that the hydrogenation of the nitro acid formed by the hydrolysis of the ethyl ester gave a rather poor yield of racemic pregabalin 1. This was even in spite of purifying the nitro acid by column chromatography. The inventors found, surprisingly, that the benzyl ester after purification and subsequent hydrogenation over palladium on carbon gave a good yield of racemic pregabalin 1.

Therefore the present invention relates to a process of preparing a γ-amino acid, comprising the step of deprotecting the ester and reducing the nitro functionality of a γ-nitro ester in one step to afford the γ-amino acid.

A particularly preferred embodiment of the process o f the present invention is outlined in scheme 2. Scheme 2 illustrates a non-limiting example of the present invention.

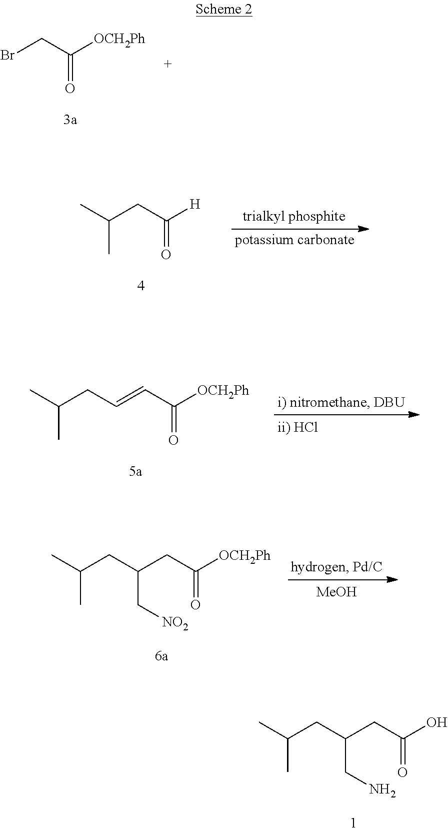

Experimental details of scheme 2 are given below.

Experimental Details

### 5-Methyl-2-hexenoic acid benzyl ester 5a

Triethyl phosphite (1 eq) and benzyl bromoacetate 3a (1 eq) were heated at 80° C. with concurrent removal of ethyl bromide for 1 hour. After the distillation was complete, the heating was stopped and isovaleraldehyde 4 (1.25 eq) was added to the cooled residue. A 50% aq. solution of potassium carbonate (2.5 eq) in water was added. The solution became turbid after 15 minutes. It was stirred for 3-4 hours at 25-30° C. and monitored by HPLC. Water was added and extracted thrice with ethyl acetate. The combined organic layers were washed with water and dried over sodium sulfate. Concentration under reduced pressure at 45-50° C. gave 5-methyl-2-hexenoic acid benzyl ester 5a in 95-99% yield as a colourless to pale yellow oil.

¹H NMR (CDCl₃, δ): 0.92 (d, 6H, J=6.65 Hz), 1.32 (m, 1H), 2.09 (m, 2H), 5.17 (s, 2H), 5.86 (d, 1H, J=15.6 Hz), 7.00 (dt, 1H, J=7.5,7.8 Hz), 7.35 (m, 5H).

¹³C NMR (CDCl₃, δ): 23.07, 28.48, 42.21, 66.68, 122.65, 128.81, 129.21, 128.85, 136.87, 149.63, 167.06.

IR (cm⁻¹, neat): 1722, 1654, 1460.

### 3-Nitromethyl-5-methyl-hexanoic acid benzyl ester 6a

To a solution of 5-methyl-2-hexenoic acid benzyl ester 5a (1 eq) in nitromethane (5 eq) at 10-15° C. was added DBU (1.05 eq) dropwise over 30 minutes. After completion of the addition, the reaction mixture was allowed to attain 25-30° C. and stirred at this temperature for 3-4 hours. After completion of the reaction, the reaction mixture was poured into cold 15% HCl and stirred for 15 minutes. The reaction mixture was extracted with ethyl acetate. The combined organic extracts were washed with water and dried over sodium sulfate. Concentration under reduced pressure gave the crude ester as a yellow oil. The crude ester was purified by column chromatography to give 3-nitromethyl-5-methyl-hexanoic acid benzyl ester 6a as pale yellow oil. Yield: 56-60%.

¹H NMR (CDCl₃, δ): 0.89 (d, 6H, J=6.50 Hz), 1.22-1.27 (t, 2H, J=7.2 Hz), 1.63 (m, 1H), 2.48 (d, 2H, J=6.41 Hz), 2.68 (m, 1H), 4.47 (m, 2H), 5.13 (s, 2H), 7.33 (m, 5H).

¹³C NMR (CDCl₃, δ): 22.95, 23.16, 25.70, 32.84, 36.70, 41.15, 67.25, 79.34, 129.01, 129.07, 129.28, 136.26, 172.03.

IR (cm⁻¹, neat): 1735, 1551, 1498.

### Racemic Pregabalin 1

Hydrogen gas was bubbled through a solution of 3-nitromethyl-5-methyl-hexanoic acid benzyl ester 6a (1 eq) in 15 volumes methanol in the presence of 60% (w/w, 50% wet) of 5% palladium on carbon. After completion of the reaction (2-3 hours), the reaction mixture was filtered through a Celite® bed. The filtrate was concentrated under reduced pressure to give racemic pregabalin 1 as an oil or sticky solid. Purification was done by crystallizing from hot 2-propanol (2 vol.) to give racemic pregabalin 1 as a white solid. Yield: 70%.

¹H NMR (D₂O, δ): 0.83 (d, 3H, J=6.48 Hz), 0.87 (d, 3H, J=6.48 Hz), 1.20 (m, 2H), 1.64 (m, 1H), 2.21 (m, 3H), 3.00 (m, 2H).

¹³C NMR (D₂O+DCl+DMSOd₆, δ): 23.39, 23.96, 26.26, 32.92, 39.26, 42.14, 45.02, 179.36.

IR (cm⁻¹, KBr): 2896, 2690, 1645.

The present invention provides an efficient synthesis of racemic pregabalin 1 from benzyl bromoacetate 3a and isovaleraldehyde 4 in three short steps, which are high yielding and afford a product which is easily purified on a commercial scale.

The difficulties encountered in the prior art for the preparation of racemic pregabalin 1 have been successfully overcome by the process of the present invention.

No trace of the troublesome lactam impurity has been observed by HPLC in the racemic pregabalin 1 or pregabalin 2, when following the process of the present invention.

It will be understood that the present invention has been described above by way of example only. The examples are not intended to limit the scope of the invention. Various modifications and embodiments can be made without departing from the scope and spirit of the invention, which is defined by the following claims only.

1-65. (canceled)

66. A process of preparing a γ-amino acid 11, comprising the step of deprotecting the ester and reducing the nitro functionality of a γ-nitro ester 16 in one step to afford the γ-amino acid 11: 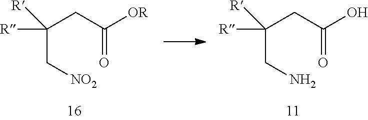
wherein R is any group that can be removed under the same reducing conditions that can convert a nitro group to an amino group, and wherein R′ and R″ are independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, or both R′ and R″ together with the carbon atom to which they are attached from a cyclic alkyl or cyclic alkenyl group, each of which may optionally be substituted, and each of which may optionally include one or more heteroatoms N, O or S in its carbon skeleton.

67. The process of claim 66, wherein:
(a) R is a benzyl, carbobenzoxy (Cbz), trityl, benzyloxymethyl, phenacyl, diphenylmethyl or 4-picolyl group, each of which may optionally be substituted;
(b) R is a benzyl, carbobenzoxy (Cbz), trityl, benzyloxymethyl, phenacyl, diphenylmethyl or 4-picolyl group, each of which is substituted with one or more nitro, halo, alkyl or alkoxy groups;
(c) R is a benzyl, substituted benzyl, carbobenzoxy (Cbz), substituted carbobenzoxy (Cbz) or trityl group;
(d) R is a benzyl group substituted with one or more nitro, halo or alkyl groups;
(e) R′ and R″ are independently hydrogen or an alkyl group, or both R′ and R″ together with the carbon atom to which they are attached from a cyclic alkyl group;
(f) R′ and R″ are independently hydrogen or a C₁₋₆ alkyl group, or both R′ and R″ together with the carbon atom to which they are attached from a C₅₋₇ cyclic alkyl group;
(g) one of R′ and R″ is hydrogen and the other is i-butyl; or
(h) both R′ and R″ together with the carbon atom to which they are attached from a cyclohexyl group.

68. The process of claim 66, wherein the deprotection of the ester and the reduction of the nitro functionality are carried out using hydrogen gas in the presence of:
(a) a catalyst;
(b) Pd/C, Pt/C or PtO₂ as catalyst; or
(c) Pd/C as catalyst.

69. The process of claim 66, wherein the γ-amino acid 11 is obtained:
(a) in a yield of 60% or more; or
(b) substantially free of lactam impurity.

70. The process of claim 66, wherein the γ-nitro ester 16 is obtained by reacting an unsaturated ester 15 with:
(a) nitromethane;
(b) nitromethane in the presence of a base; or
(c) nitromethane in the presence of DBU as base.

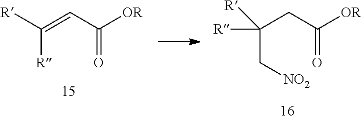

71. The process of claim 70, wherein the unsaturated ester 15 is obtained by reacting an aldehyde or ketone 14 with:
(a) a phosphonoacetate;
(b) a phosphonoacetate in the presence of a base; or
(c) a phosphonoacetate in the presence of potassium carbonate as base.

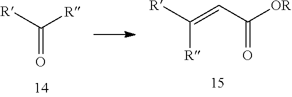

72. The process of claim 71, wherein the phosphonoacetate 9 is prepared in situ from a trialkyl phosphite 8 and an acetic acid ester 3: 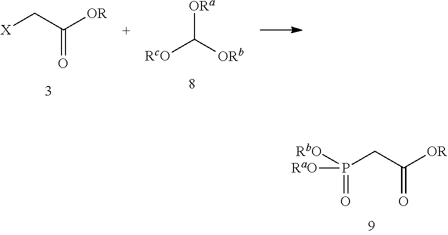
wherein:
(a) X is a leaving group, and R^{a}, R^{b} and R^{c} are independently alkyl groups;
(b) X is a halo or sulfonate group, and R^{a}, R^{b} and R^{c} are independently alkyl groups;
(c) X is a chloro, bromo or iodo group, and R^{a}, R^{b} and R^{c} are independently alkyl groups;
(d) X is a bromo group, and R^{a}, R^{b} and R^{c} are independently alkyl groups; or
(e) X is a bromo group, R is a benzyl group, and R^{a}, R^{b} and R^{c} are ethyl groups.

73. The process of claim 66, wherein R′ and R″ are not the same, wherein the γ-amino acid 11 is racemic, and wherein the process further comprises the step of resolving the racemic γ-amino acid 11.

74. A γ-amino acid 11, prepared by the process of claim 66, which is:
(a) racemic or optically inactive; or
(b) enantiomerically pure or enantiomerically enriched, and prepared by the process further comprising the step of resolving the racemic γ-amino acid.

75. A pharmaceutical composition comprising the γ-amino acid of claim 74.

76. A process of preparing racemic pregabalin 1, comprising the step of deprotecting the ester and reducing the nitro functionality of a 3-nitromethyl-5-methyl-hexanoic acid ester 6 in one step to afford racemic pregabalin 1: 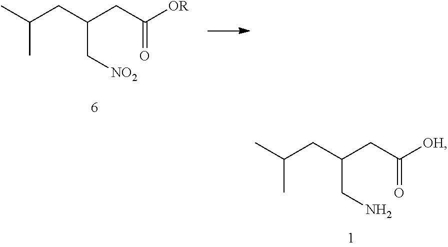
wherein R is any group that can be removed under the same reducing conditions that can convert a nitro group to an amino group.

77. The process of claim 76, wherein:
(a) R is a benzyl, carbobenzoxy (Cbz), trityl, benzyloxymethyl, phenacyl, diphenylmethyl or 4-picolyl group, each of which may optionally be substituted;
(b) R is a benzyl, carbobenzoxy (Cbz), trityl, benzyloxymethyl, phenacyl, diphenylmethyl or 4-picolyl group, each of which is substituted with one or more nitro, halo, alkyl or alkoxy groups;
(c) R is a benzyl, substituted benzyl, carbobenzoxy (Cbz), substituted carbobenzoxy (Cbz) or trityl group; or
(d) R is a benzyl group substituted with one or more nitro, halo or alkyl groups.

78. The process of claim 76, wherein the deprotection of the ester and the reduction of the nitro functionality are carried out using hydrogen gas in the presence of:
(a) a catalyst;
(b) Pd/C, Pt/C or PtO₂ as catalyst; or
(c) Pd/C as catalyst.

79. The process of claim 76, wherein the racemic pregabalin 1 is obtained:
(a) in a yield of 60% or more; or
(b) substantially free of lactam impurity.

80. The process of claim 76, wherein the 3-nitromethyl-5-methyl-hexanoic acid ester 6 is obtained by reacting an ester of 5-methyl-2-hexenoic acid 5 with:
(a) nitromethane;
(b) nitromethane in the presence of a base; or
(c) nitromethane in the presence of DBU as base.

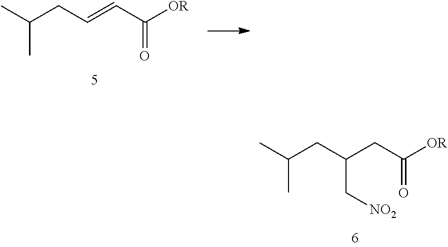

81. The process of claim 80, wherein the 5-methyl-2-hexenoic acid ester 5 is obtained by reacting isovaleraldehyde 4 with:
(a) a phosphonoacetate;
(b) a phosphonoacetate in the presence of a base; or
(c) a phosphonoacetate in the presence of potassium carbonate as base.

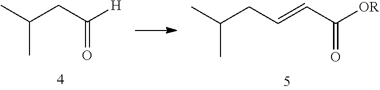

82. The process of claim 81, wherein the phosphonoacetate 9 is prepared in situ from a trialkyl phosphite 8 and an acetic acid ester 3: 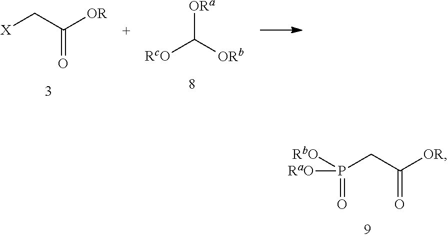
wherein:
(a) X is a leaving group, and R^{a}, R^{b} and R^{c} are independently alkyl groups;
(b) X is a halo or sulfonate group, and R^{a}, R^{b} and R^{c} are independently alkyl groups;
(c) X is a chloro, bromo or iodo group, and R^{a}, R^{b} and R^{c} are independently alkyl groups;
(d) X is a bromo group, and R^{a}, R^{b} and R^{c} are independently alkyl groups; or
(e) X is a bromo group, R is a benzyl group, and R^{a}, R^{b} and R^{c} are ethyl groups.

83. Racemic pregabalin 1, which is prepared by the process of claim 76.

84. A process of preparing pregabalin 2, wherein the process comprises the process of preparing racemic pregabalin 1 as claimed in claim 76.

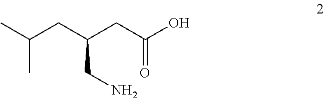

85. Pregabalin 2, which is prepared by the process of claim 84.
